(19) United States
(12) Patent Application Publication
(10) Pub. No.: US 2006/0116354 A1
(43) Pub. Date: Jun. 1, 2006

(54) Antineoplastic ether lipid compounds with modifications at the sn-3 carbon

(76) Inventor: Walter R. Perkins, Pennington, NJ (US)

Correspondence Address: NIXON & VANDERHYE, PC, 901 NORTH GLEBE ROAD, 11TH FLOOR, ARLINGTON, VA, 22203, US

(21) Appl. No.: 10/541,864

(22) PCT Filed: Jan. 9, 2004
(86) PCT No.: PCT/US04/00358

§ 371 Date: Nov. 10, 2005

(30) Foreign Application Priority Data
Jan. 9, 2003 (US) 60438785

(51) Int. Cl. A61K 31/685; C07F 9/547

(52) U.S. Cl. 514/78; 544/157; 546/22; 548/413; 554/76

(57) ABSTRACT

Ether lipid compounds of formula (I), pharmaceutically-acceptable salts, prodrugs or isomers thereof are provided, where the variables are as defined herein. The compounds of the invention have anti-neoplastic activity, and accordingly have utility in treating cancer and related diseases. Enantiomers of these compounds, pharmaceutical compositions, and methods for treating cancer with the pharmaceutical compositions are also provided.

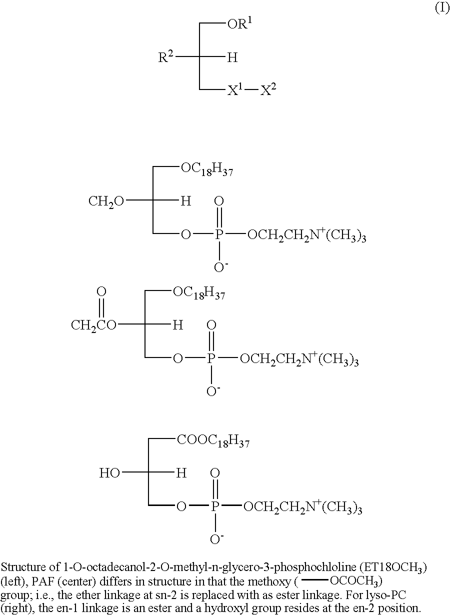

### BACKGROUND OF THE INVENTION

Field of the Invention

The present invention provides novel ether lipid compounds with modifications at the sn-3 carbon, pharmaceutically-acceptable salts, prodrugs or isomers thereof, as well as pharmaceutical compositions, and methods for treating cancer.

### REFERENCES

The following publications, patents and patent applications are cited in this application as superscript numbers:
¹ Berdel, W. E., Br. J. Cancer, 64:208-211 (1991).
² Lohmeyer, M. and Bittman, R., Drugs of the Future, 19:1021-1037 (1994).
³ Berdel, W. E. and Munder, P. G. in Platelet Activating Factor and Related Lipid Mediators, (F. Snyder, Ed.), pp. 449-468, Plenum Press, New York, N.Y. (1987).

⁴ Houlihan, W. J., Lohmeyer, M., Workman, P. and Cheon, S. H., Med Res. Rev., 15:157-223 (1995).
⁵ Principe, P. and Braquet, P., Rev. Oncol. Hematol., 18:155-178 (1995).
⁶ Berdel, W. E., Bausert, W. R. E., Fink, U., Rastetter, J. and Munder, P. G., Anticancer Res. 1, 345-352 (1981).
⁷ Bittman, R. in Phospholipids Handbook: Chemical Preparation of Glycerolipids, (Cevc, G. Ed.), Marcel Dekker, Inc., New York, N.Y., pp. 141-232 (1993), and references therein.

⁸ Berdel, W. E., Andreesen, R., and Munder, P. G. in Phospholipids and Cellular Regulation, Vol 2. (Kuo, J, Ed), CRC Press: Boca Raton, Fla., pp. 41-73 (1985).
⁹ Boggs, K. P., Rock, C. O. and Jackowski, S., J. Biol. Chem., 270: 11612-11618 (1995).

¹⁰ Boggs, K. P., Rock, C. O. and Jackowski, S., Biochim. Biophys. Acta, 1389:1-12 (1998).
¹¹ Geilen, C. G., Weider, T., Geilen, C. C., Reutter, W., J. Biol. Chem., 267:6719-6724 (1992).
¹² Gajate, C., Santos-Beneit, A., Modolell, M. and Mollinedo, F., Mol. Pharmacol., 53:602-612 (1998).
¹³ Ruiter, G. A., Zerp, S. F., Bartelink, H., Van Blitterswijk, W. J. and Verheij, M., Cancer Res. 59, 2457-2463 (1999).
¹⁴ Wieder, T., Orfanos, C. E. and Geilen, C. G., J. Biol. Chem., 273: 11025-11031 (1998).
¹⁵ Bittman, R., and Arthur, G. in Liposomes: Rational Design (A. S. Janoff, Ed.) pp 125-144, Marcel Dekker, New York, N.Y. (1998).
¹⁶ Arthur, G. and Bittman, R., Biochim. Biophys. Acta. 1390:85-102 (1998).
¹⁷ Andreseen, R., Osterholz, J., Luckenbach, G. A., Costabel, U., Schulz, A., Speth, V., Munder, P. G. and Lohr, G. W., J. Natl. Cancer Inst., 72:53-59 (1984).
¹⁸ Yamamoto, N. and Ngwenya, B. Z., Cancer Res. 47:2008-2013 (1987).
¹⁹ Heesbeen, E. C., Verdonck, L. F., Hermans, S. W. G., van Heugten, H. G., Staal, G. E. J., Rijksen, G., FEBS Lett., 290:231-234 (1991).
²⁰ Honman, Y., Kasukabe, T., Hozumi, M., Tsushima, S., Nomura, H., Cancer Res., 46:5803-5809 (1980).
²¹ Zhou, X., Lu, X., Richard, C., Xiong, W., Litchfield, D. W., Bittman, R. and Arthur, G., J. Clin. Invest. 98:937-944 (1996).
²² Marshall, C. J., Cell, 80:179-185 (1995).
²³ Mayhew, E., Ahmad, I., Bhatia, S., Dause, R., Filep, J., Janoff, A. S., Kaisheva, E., Perkins, W. R., Zha, Y., Franklin, J. C., Biochim. Biophys. Acta, 1329, 139-148 (1997).
²⁴ Powis, G., Seewald, M. J., Gratas, C., Melder, D., Riebow, J., Modest, E. J., Cancer Res., 52:2835-2840 (1992).
²⁵ Wilcox, R. W., Wykle, R. L., Schmitt, J. D. and Daniel, L. W., Lipids, 22:800-807 (1987).
²⁶ Bishop, F. E., Dive, C., Freeman, S. and Gescher, A., Cancer Chemother. Pharmacol., 31:85-92 (1992).
²⁷ Cuvillier, O., Mayhew, E., Janoff, A. S. and Spiegel, S., Blood, 94: 3583-3592 (1999).
²⁸ Kim, U. T., Bhatia, S. K. and Hajdu, J., Tetrahedron Lett., 32:6521-6524 (1991).
²⁹ Ahmad, I., Filep, J. J., Franklin, J. C., Janoff, A., Masters, G. R., Pattassery, J., Peters, Schupaky, J. J., Zho, Y. and Mayhew, E.; Cancer Res., 15:1915-1921 (1997).
Peters, A. P., Ahmad, I., Janoff, A. S., Pushkerava, M. Y., Mayhew, E., Lipids, 32:1045-1054 (1997).
³¹ Volger, W. R., Whigham, E., Bennette, W. and Olson, A. C., Exper. Hemato., 13:629-633 (1985).
³² Zheng, B., Ioshi, K., Shoji, M., Eibl, H., Berdel, W. E., Hajdu, J., Vogler, W. R., Kuo, J. F., Cancer Res., 50:3025-3032 (1990).
³³ Kotting, J. and Eibl, H. in Lipases, Their Structure, Biochemistry and Application (Peterson, S R and Wooley, P. Eds.), pp. 289, Cambridge University Press, Cambridge, Mass. (1994).
³⁴ Guivisdalsky, P. N. and Bittman, R., Tetrahedron Lett., 29:4393-4396 (1988).
³⁵ Abdelmageed, O. S., Duclos, R. I., Abushanab, E., Makriyannis, A., Chem. Phys. Lipids, 54:49-59 (1990).
³⁶ Ali, S. and Bittman, R.; Biochem. Cell Biol., 68:360-365 (1990).
³⁷ Teraji et al., “Phospholipid Derivatives, and Pharmaceutical Composition of the Same,” U.S. Pat. No. 4,562,179, issued Dec. 31, 1985.
³⁸ Pinchul, A. N., Mistner, B. I. and Shvets, V. I., Chem. Phys. Lipids, 65:65-75 (1993).

All of the above publications, patents and patent applications are herein incorporated by reference in their entirety to the same extent as if each individual publication, patent or patent application was specifically and individually indicated to be incorporated by reference in its entirety.

### STATE OF THE ART

Alkyllysophospholipids (AALPs) and allylphosphocholines (APCs) represent subclasses of potential antitumor agents collectively known as antitumor ether lipids (AELs). They do not interact with cellular DNA and are therefore not mutagenic.¹⁻³ The antitumor activities of these compounds, which are based on lysophosphatidylcholine, are now established. The prototype of the alkyllysophospholipids (ALPs), 1-O-octadecyl-2-O-methyl-glycerophosphocholine (ET-18-OCH₃), and other ether-linked phosphocholine analogues are in clinical trials.^{2, 4-7} Compound ET-18-OCH₃, a subclass of alkyl lysophospholipids (ALPs), is known for its anti-cancer activities against breast (MCF-7), Lewis lung (A549), and ovarian (Ovcar-3) cell lines.^{2,4} Structurally similar, the platelet activating factor (PAF) differs from ET-18-OCH₃ merely in an ester linkage at the sn-2 position of the glycerol-backbone. Both PAF and ET-18-OCH₃ are known to inhibit protein kinase C activity and phosphatidylcholine choline biosynthesis.^{3, 31, 32} ALPs also appear to inhibit the proliferation of tumor cells without affecting the growth of normal cells.⁸ While the mechanism of inhibition of cell proliferation has yet to be resolved, various hypotheses have been proposed. In some cells, ALPs and APCs appear to induce apoptosis as a consequence of inhibition of phosphatidylcholine synthesis.⁹⁻¹¹ Other theories for the mechanism of action include activation of the stress activated protein kinase pathways,¹²⁻¹³ drug-induced increase in cellular ceramide levels,¹⁴ nutrient starvation, inhibition of transacylase activity, enhanced lipid peroxidation, inhibition of cellular signaling pathways¹⁵⁻¹⁶ and/or activation of tumoricidal macrophages.¹⁷⁻¹⁸

Other studies have revealed that ALPs affect the activity of a large number of signaling molecules including protein kinase C (PKC), phosphatidylinositol 3-kinase, phosphatidylinositol-specific phospholipase C, and diacylglycerol kinase.^{19, 20, 16} Recently another signaling molecule, Raf-1, was added to the list with the demonstration that ET-18-OCH₃′ decreased the levels of Raf-1 associating with the cell membrane in growth-factor stimulated MCF-7 cells which consequently led to decreased activation of MAP kinase,²¹ a crucial enzyme required in initiating cell proliferation.²² It was suggested that Raf-1 is a primary target of ALPs in cells. The large number of molecules affected by ALPs has complicated the task of separating their primary site(s) of action from secondary events.

The finding that the glycerol-based ether lipids possess anti-neoplastic activities, has led investigators to explore isosteric and isoformic analogs of ET-18-OCH₃ especially in areas of synthesis, biological and biophysical properties.⁶⁻⁷ ET-18-OCH₃ formulated in liposomes (ELL-12), is currently being evaluated in Phase I clinical trial.²⁹⁻³⁰

Despite the progress that has been made in understanding the underlying mechanisms of antitumor ether lipids, there remains a need to develop novel compounds and compositions for the treatment of disease. Ideally, the treatment methods would advantageously be based on ether lipids that are capable of acting as anti-neoplastic agents.

### SUMMARY OF THE INVENTION

The invention is directed to the discovery of a class of anti-tumor ether lipid compounds having anti-neoplastic activity. Preferably, the invention provides bioactive ether lipid compounds with modifications at the sn-3 carbon or pharmaceutically-acceptable salts, prodrugs or isomers thereof. The invention also relates to pharmaceutical compositions comprising these compounds, and methods for treating cancer.

In one embodiment, the invention relates to an ether lipid having formula (I), or a pharmaceutically acceptable salt, isomer or prodrug thereof: 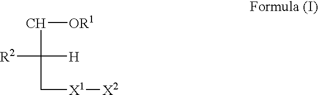
R¹ is selected from the group consisting of alkyl, alkenyl and alkynyl.
R² is selected from the group consisting of —OR³.
R³ is selected from the group consisting of C₁₋₄ alkyl and H.
X¹ is selected from the group consisting of —OSO₂(CH₂)ₘ—, —OSO₂NR⁴(CH₂)ₘ, 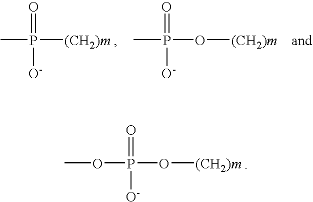
X² is selected from the group consisting of: 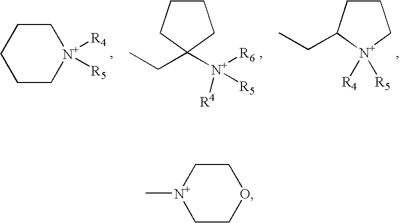 CH₂(CH₂) N⁺R⁴, R⁵, R⁶ and —CH₂(CH₂)ₚP⁺R⁷R⁸R⁹.
R⁴, R⁵ and R⁶ are each independently selected from the group consisting of H and C₁₋₁₀ alkyl. R⁷, R⁸ and R⁹ are each independently a C₁₋₃alkyl group; and m and p are each independently 0 or an integer from 1-10.

Preferably, R¹ is represented by Y¹Y², wherein:

Y¹ is (CH)n₁(CH═CH)n₂(CH═CH)n₃(CH═CH)n₄(CH₂)n₅(CH═CH)n₆(CH₂)n₇ (CH═CH)n₈(CH₂)n₉, the sum of n₁+2n₂+n₃+2n₄ is equal to n₅+2n₆+n₇n+2n₈+n₉ is an integer of from 3 to 23, n, is zero or an interger of from 1 to 22, n₃ is zero or an integer of from 1 to 19, n₅ is zero or an integer of from 1 to 16, n₇ is zero or an integer of from zero to 16, n₉ is zero or an integer of from 1 to 10, and each of n₂, n₄, n₆ and n₈ is independently zero or 1; and
Y₂ is —CH₃ or —CO₂H.

Preferably, R¹ is selected from the group consisting of —C₁₈H₃₇ and —C₁₆H₃₃.

Preferably, R³ is Me.

Preferably, X¹ is —OSO₂(CH₂)ₘ— 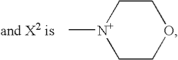
or X¹ is 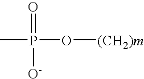
and X² is CH₂(CH₂)N⁺R⁴, R⁵, R⁶, particularly when m is an integer from 1-5 and/or R⁴, R⁵ and R⁶ are each a methyl group.

Preferably, 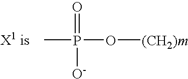
and X² is —CH₂(CH₂)ₚR⁺R⁴R⁵R⁶, particularly when m is 0 and p is an integer from 1-8 and/or R⁴, R⁵ and R⁶ are each a methyl group.

Preferred compounds of Formula (I) include the following:

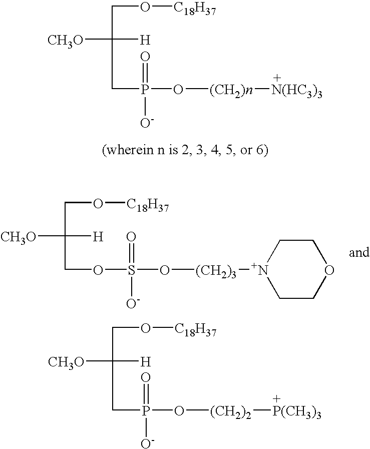

Preferably, the compound of Formula (I) is optically active, more preferably, the compound of Formula (I) is the D enantiomer.

In a preferred embodiment, the compounds according to the invention will not aggregate platelets (i.e., mimic PAF). The chemical structure of PAF platelet aggregation factor) is shown in FIG. 1. In an embodiment of the invention, the antitumor ether lipid compounds will avoid PAF recognition while maintaining or enhancing activity and selectivity. However, in those cases where a platelet aggregation response to the antitumor ether lipid compounds is observed, co-administration with a PAF antagonist may be used to block such a response. In yet another embodiment of the invention, the D isomer is used in order to avoid a platelet aggregation response.

In a further embodiment of the invention, the antineoplastic ether lipid compounds will (1) inhibit growth of tumor cells, and (2) inhibit growth of normal cell lines as compared to tumor cells. Further, it is also preferred that the compounds of the invention will not aggregate platelets, will not lyse red blood cells and have desirable pharmacokinetic properties.

Additionally, the invention relates to pharmaceutical compositions comprising a pharmaceutically acceptable carrier and a pharmaceutically effective amount of a compound of formula (I). The pharmaceutical compositions may comprise (a) a liposome, emulsion or mixed miscelle carrier and (b) a pharmaceutically effective amount of compound of formula (I) or a pharmaceutically acceptable salt, isomer or prodrug thereof. The invention further relates to a liposome comprising a compound of formula (I) or a pharmaceutically acceptable salt, isomer or prodrug thereof.

These pharmaceutical compositions can be used in methods for treating a mammal afflicted with a cancer, comprising administering to the mammal a therapeutically effective amount of the pharmaceutical composition. Typical dosages range from about 0.1 to about 1000 mg of the compound of formula (I) per kg of the body weight of the mammal per day.

The type of cancer to be treated may be selected from the group consisting of, but not limited to: lung cancers, brain cancers, colon cancers, ovarian cancers, breast cancers, leukemias, lymphomas, sarcomas, and carcinomas.

The treatment methods according to the invention may also include administering to the mammal an additional biologically active agent. Any suitable biologically active agent may be used in combination with the ether lipids of the invention. In a preferred embodiment, the additional biologically active agent may be selected from the group consisting of antineoplastic agents, antimicrobial agents, and hematopoietic cell growth stimulating agents.

### BRIEF DESCRIPTION OF THE DRAWINGS

FIG. 1 depicts the structure of 1-O-octadecanol-2-O-methyl-sn-glycero-3-phosphocholine (ET-18-OCH₃) (left), PAF (center) and lyso-PC (right). PAF differs in structure in that the methoxy (—OCH₃) is replaced with an acetyl (—OCOCH₃) group; i.e., the ether linkage at sn-2 is replaced with an ester linkage. For lyso-PC, the sn-1 linkage is an ester and a hydroxyl group resides at the sn-2 position.

FIG. 2 depicts a scheme for the synthesis of compounds of the invention.

FIG. 3 depicts a scheme for the synthesis of compounds of the invention.

FIG. 4 depicts a scheme for the synthesis of compounds of the invention.

FIG. 5 depicts a scheme for the synthesis of compounds of the invention.

FIG. 6 depicts a scheme for the synthesis of compounds of the invention.

FIG. 7 depicts growth inhibitory effects of new ether lipids against normal human (WI-38) and murine (NIH-3T3) fibroblast cell lines and the human colon tumor cell line HT29. L-ET-18-OCH₃ and D-ET-18-OCH₃ are shown for comparison. The values used are the larger of the numbers when repeat experiments were performed.

FIG. 8A-I depicts GI₅₀ values for compounds sent for testing at NCI's Drug Discovery Program for screening against numerous human tumor cell lines (renal, ovarian, colon, CNS, non-small cell lung, leukemia, breast, melanoma and prostate.)

### DETAILED DESCRIPTION OF THE INVENTION

As stated above, this invention relates to novel ether lipid compounds, pharmaceutically-acceptable salts, prodrugs, or isomers thereof, which have utility as anti-neoplastic agents. In particular, the invention relates to ether lipid compounds of formula (I), having modifications at the sn-3 carbon. However, prior to describing this invention in further detail, the following terms will first be defined.

Definitions

The term “alkyl” refers to saturated aliphatic groups including straight-chain, branched-chain, cyclic groups, and combinations thereof. The alkyl groups preferably have between 1 to 20 carbon atoms.

The term “alkenyl” refers to unsaturated aliphatic groups including straight-chain, branched-chain, cyclic groups, and combinations thereof, having at least one double bond and having the number of carbon atoms specified. The alkenyl groups preferably have between 1 to 20 carbon atoms.

The term “cyclic alkyl” or “cycloalkyl” refers to alkyl group forming an aliphatic ring. Preferred cyclic alkyl groups have about 3 carbon atoms.

The term “direct link” as used herein refers to a bond directly linking the substituents on each side of the direct link.

The ether lipids of the invention have a 3 carbon alcohol, glycerol, as the backbone. With the 3 carbons of glycerol, positions are designated as stereospecific numbers, sn, to distinguish location. The designations “sn-1,” “sn-2,” and “sn-3” identify glycerol carbons 1, 2, and 3, respectively. The glycerol carbons are labeled below for formula (I):

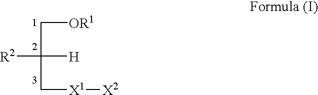

“Pharmaceutically acceptable salt” refers to pharmaceutically acceptable salts that are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like. Examples of pharmaceutically acceptable acid addition salts includes salts which retain the biological effectiveness and properties of the free bases and which are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Examples of pharmaceutically acceptable base addition salts include those salts derived from inorganic bases such as sodium, potassium, lithium, ammonium, calcium, magnesium, iron, zinc, copper, manganese, and aluminum bases, and the like. Particularly preferred are the ammonium, potassium, sodium, calcium and magnesium salts. Salts derived from pharmaceutically acceptable organic nontoxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like. Particularly preferred organic nontoxic bases are isopropylamine, diethylamine, ethanolamine, trimethamine, dicyclohexylamine, choline, and caffeine.

“Prodrug” means any compound which releases an active parent drug according to formula (I) in vivo when such prodrug is administered to a mammalian subject. Prodrugs of a compound may be prepared by modifying functional groups present in the compound in such a way that the modifications may be cleaved in vivo to release the parent compound. Prodrugs include compounds of formula (I) wherein a hydroxy, amino, or sulfhydryl group is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), carbamates (e.g., N,N-dimethylamino-carbonyl), and the like.

“Isomers” are compounds that have the same molecular formula but differ in the nature or sequence of bonding of their atoms or the arrangement of their atoms in space. Isomers that differ in the arrangement of their atoms in space are termed “stereoisomers.” Stereoisomers that are not mirror images of one another are termed “diastereomers” and those that are non-superimposable mirror images of each other are termed “enantiomers.” An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Treating” or “treatment” of a disease includes:

(1) preventing the disease, i.e. causing the clinical symptoms of the disease not to develop in a mammal that may be exposed to or predisposed to the disease but does not yet experience or display symptoms of the disease,

(2) inhibiting the disease, i.e., arresting or reducing the development of the disease or its clinical symptoms, or

(3) relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

A “pharmaceutically acceptable carrier” means an carrier that is useful in preparing a pharmaceutical composition that is generally safe, non-toxic and neither biologically nor otherwise undesirable, and includes a pharmaceutically acceptable excipient that is acceptable for veterinary use or human pharmaceutical use. A “pharmaceutically acceptable excipient” as used in the specification and claims includes both one and more than one such excipient. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

“Cancer” refers to a group of diseases characterized by uncontrolled growth and spread of abnormal cells, often resulting in the formation of a non-structured mass or tumor. Illustrative tumors include carcinomas, sarcomas and melanomas, such as basal cell carcinoma, squamous cell carcinoma, melanoma, soft tissue sarcoma, solar keratoses, Kaposi's sarcoma, cutaneous malignant lymphoma, Bowen's disease, Wilm's tumor, hepatomas, colorectals cancer, brain tumors, mycosis fungoides, Hodgkin's lymphoma, polycythemia vera, chronic granulocytic leukemia, lymphomas, oat cell sarcoma, and the like. Tumors may also include benign growths such as condylomata acuminatal (genital warts) and moles and common warts.

An “anti-neoplastic agent” is a pharmaceutical which inhibits or causes the death of cancer or tumor cells.

An “antimicrobial agent” is a substance that either destroys or inhibits the growth of a microorganism at concentrations tolerated by the infected host.

A “hematopoietic cell growth stimulating agent” is one that stimulates blood cell growth and development, i.e. of red blood cells, leukocytes, and platelets. Such agents are well known in the art. For example, in order to increase infection-fighting white blood cell production, recombinant granulocyte-colony stimulating factor may be used to stimulate the growth of neutrophils. Another example of a hematopoietic cell growth stimulating agent is recombinant granulocyte macrophage-colony stimulating factor, which increases production of neutrophils, as well as other infection-fighting white blood cells, granulocytes and monocytes, and macrophages. Another hematopoietic agent is recombinant stem cell factor, which regulates and stimulates the bone marrow, specifically to produce stem cells.

Compound Preparation

The compounds of formula (I) can also be prepared via several divergent synthetic routes with the particular route selected relative to the ease of compound preparation, the commercial availability of starting materials, and the like. For instance, the compounds of formula (I) may be synthesized and tested using the methods exemplified in the examples and the instant specification. Such methods may be further adapted to produce analogs, derivatives and variants within the scope of formula (I).

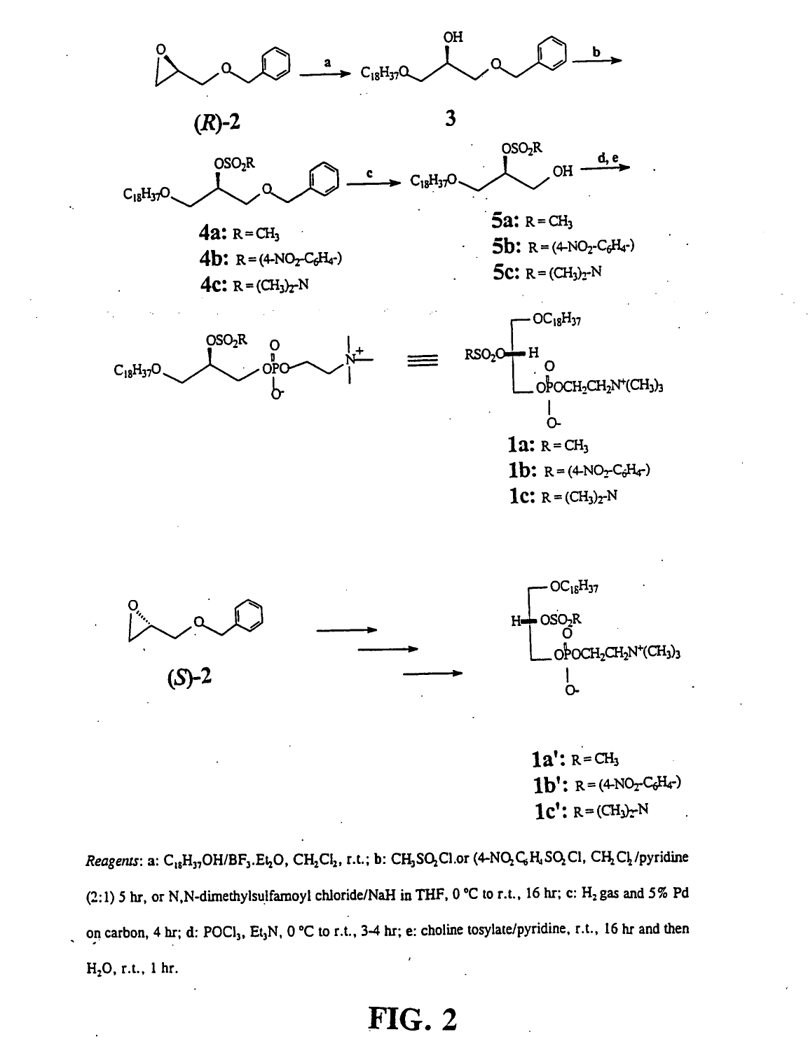

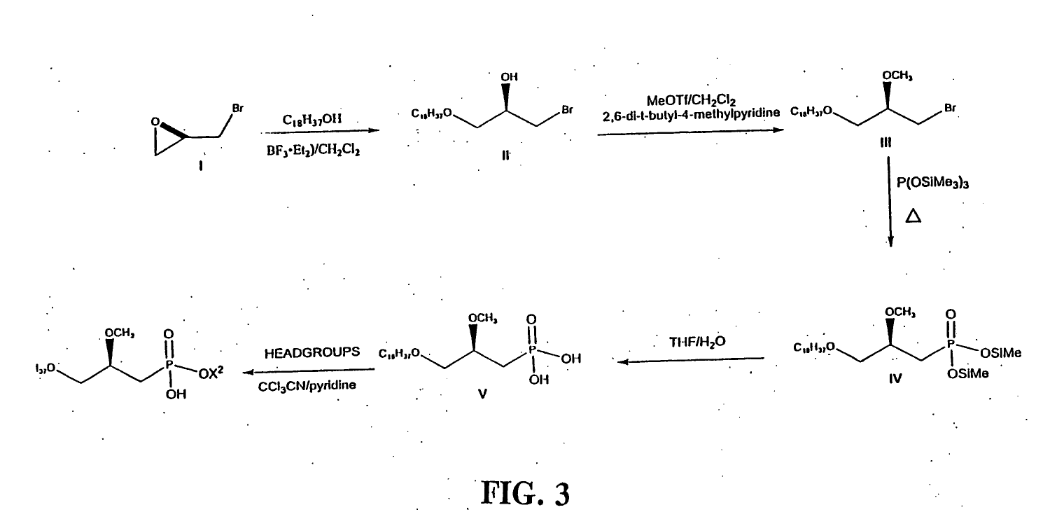

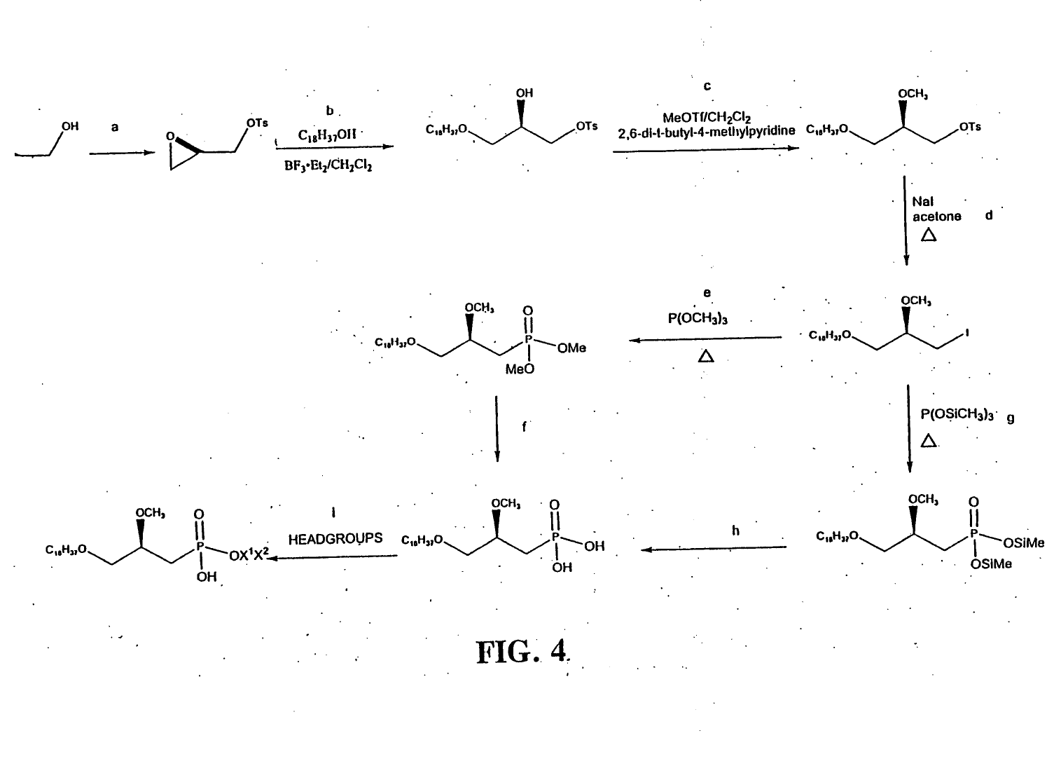

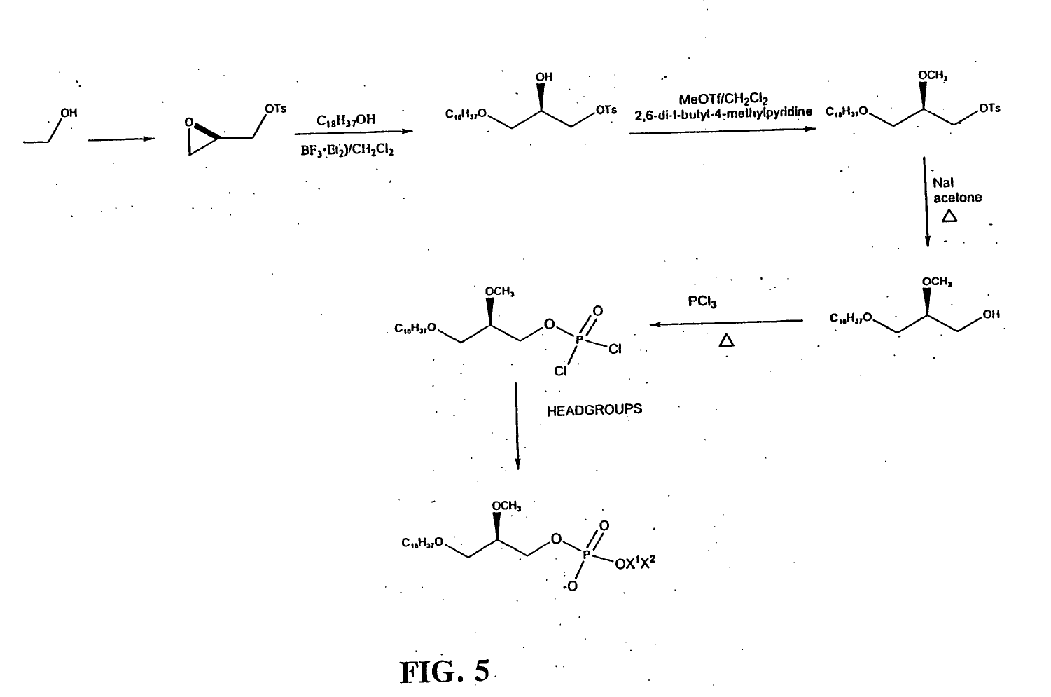

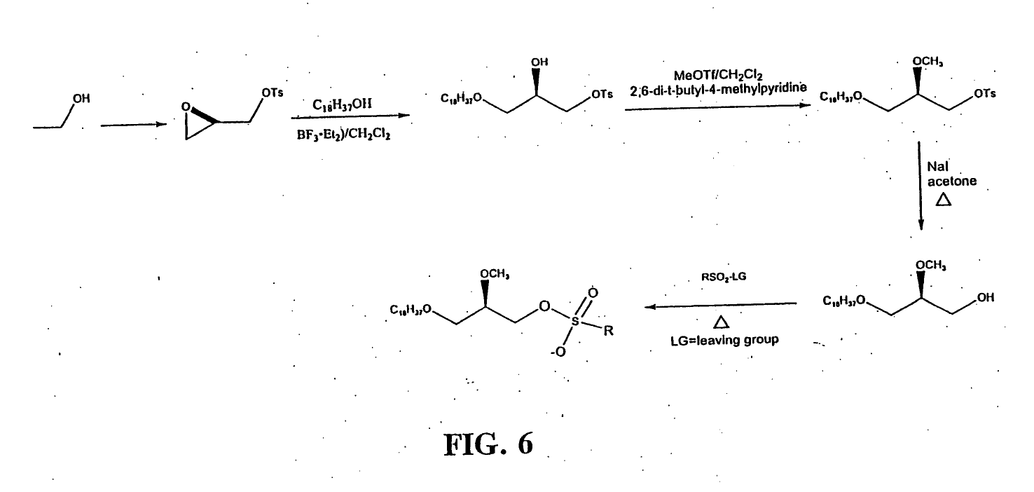

A general route to the synthesis of the compounds of formula (I) is shown in FIG. 2. As shown in FIG. 2, to a stirred solution of the alcohol, e.g. 1-octadecanol and (S)-4-bromo-1,2-epoxypropane (1) in anhydrous CH₂Cl₂, was added BF₃ etherate. Other alcohols may be used in place of 1-octadecanol, depending upon what substitution is desired at the sn-1 position. The reaction mixture was stirred for about 18 hr or until the reaction was complete under nitrogen atmosphere. After the reaction is complete, the solvent is removed under reduced pressure, the resulting white crude solid is purified via column chromatography or other suitable means to yield the alcohol (II).

To the alcohol (II) in anhydrous CH₂Cl₂, was added 2,6-ditertbutyl-4 methyl-pyridine. After 5 min of stirring, methyl triflate was added and the reaction was continued at reflux at 40° C. for 16 hr. Reaction was monitored by TLC. After the reaction was complete, the solvent was removed under reduced pressure. The resulting residue is taken up in ethyl acetate and washed with 2M HCl (2×100 ml), 5% NaHCO₃, and dried over Na₂SO₄. Solvent filtration and removal under reduced pressure resulted in a white solid, which is purified by column chromatography or any other suitable technique to yield methyl ether (III).

A solution of compound (III) and excess of tris(trimethylsilyl) phosphite is heated at 125° C. for 24 hr to yield IV. The unreacted phosphite and bromotrimethylsilane are removed, e.g. via vacuum distillation at ≦90° C. After the distillation, the crude bis-silylphosphite IV was cooled to room temperature and was subjected to hydrolysis in THF:H₂O (9:1) at room temperature for about 12 hr or until the reaction is complete to yield V. The crude waxy white product was dried and used in the next step without further purification.

Compound V may then be derivatized with a variety of headgroups to yield compounds of formula (I). For instance, in a typical procedure, trichloroacetonitrile, and 2-triethylaminoethanol tosylate are added to compound V in anhydrous pyridine.

The solution is heated to about 50° C. for 48 hrs under N₂ atmosphere or until the reaction is complete. Solvent is then removed under reduced pressure. The product may be isolated and purified using standard techniques known in the art.

Pharmaceutical Formulations

When employed as pharmaceuticals, the compounds of formula (I) are usually administered in the form of pharmaceutical compositions. These compounds can be administered by a variety of routes including oral, rectal, transdermal, subcutaneous, intravenous, intramuscular, and intranasal. These compounds are effective as both injectable and oral compositions. Such compositions are prepared in a manner well known in the pharmaceutical art and comprise at least one active compound.

This invention also includes pharmaceutical compositions which contain, as the active ingredient, one or more of the compounds of formula (I) above associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Preferably, the compound of formula (I) above is employed at no more than about 20 weight percent of the pharmaceutical composition, more preferably no more than about 15 weight percent, with the balance being pharmaceutically inert carrier(s).

The active compound is effective over a wide dosage range and is generally administered in a pharmaceutically effective amount. It will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices which deliver the formulation in an appropriate manner. The following formulation examples illustrate representative pharmaceutical compositions of the present invention.

### Formulation Example 1

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules in 340 mg quantities.

### Formulation Example 2

A tablet formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### Formulation Example 3

A dry powder inhaler formulation is prepared containing the following components:

| Ingredient | Weight % |
|---|---|
| Active Ingredient | 5 |
| Lactose | 95 |

The active ingredient is mixed with the lactose and the mixture is added to a dry powder inhaling appliance.

### Formulation Example 4

Tablets, each containing 30 mg of active ingredient, are prepared as follows:

| | Quantity | |
|---|---|---|
| Ingredient | (mg/tablet) | |
| Active Ingredient | 30.0 | mg |
| Starch | 45.0 | mg |
| Microcrystalline cellulose | 35.0 | mg |
| Polyvinylpyrrolidone (as 10% solution in sterile water) | 4.0 | mg |
| Sodium carboxymethyl starch | 4.5 | mg |
| Magnesium stearate | 0.5 | mg |
| Talc | 1.0 | mg |
| Total | 120 | mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### Formulation Example 5

Capsules, each containing 40 mg of medicament are made as follows:

| | Quantity | |
|---|---|---|
| Ingredient | (mg/capsule) | |
| Active Ingredient | 40.0 | mg |
| Starch | 109.0 | mg |
| Magnesium stearate | 1.0 | mg |
| Total | 150.0 | mg |

The active ingredient, starch, and magnesium stearate are blended, passed through a No. 20 mesh U.S. sieve, and filled into hard gelatin capsules in 150 mg quantities.

### Formulation Example 6

Suppositories, each containing 25 mg of active ingredient are made as follows:

| Ingredient | Amount | |
|---|---|---|
| Active Ingredient | 25 | mg |
| Saturated fatty acid glycerides | 2,000 | mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation Example 7

Suspensions, each containing 50 mg of medicament per 5.0 mL dose are made as follows:

| Ingredient | Amount | |
|---|---|---|
| Active Ingredient | 50.0 | mg |
| Xanthan gum | 4.0 | mg |
| Sodium carboxymethyl cellulose (11%) | | |
| Microcrystalline cellulose (89%) | 50.0 | mg |
| Sucrose | 1.75 | g |
| Sodium benzoate | 10.0 | mg |
| Flavor and Color | q.v. | |
| Purified water to | 5.0 | mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Formulation Example 8

| | Quantity | |
|---|---|---|
| Ingredient | (mg/capsule) | |
| Active Ingredient | 15.0 | mg |
| Starch | 407.0 | mg |
| Magnesium stearate | 3.0 | mg |
| Total | 425.0 | mg |

The active ingredient, starch, and magnesium stearate are blended, passed through a No. 20 mesh U.S. sieve, and filled into hard gelatin capsules in 425.0 mg quantities.

### Formulation Example 9

A subcutaneous formulation may be prepared as follows:

| Ingredient | Quantity |
|---|---|
| Active Ingredient | 5.0 mg |
| Corn Oil | 1.0 mL |

### Formulation Example 10

A topical formulation may be prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference in its entirety. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472 which is herein incorporated by reference in its entirety.

Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate- and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

Other suitable formulations for use in the present invention can be found in Remington's Pharmaceutical Sciences, Mace Publishing Company, Philadelphia, Pa., 17th ed. (1985), which is hereby incorporated by reference in its entirety.

Utility

The compounds and pharmaceutical compositions of the invention are useful as anti-neoplastic agents, and accordingly, have utility in treating cancer in mammals including humans.

As noted above, the compounds described herein are suitable for use in a variety of drug delivery systems described above. Additionally, in order to enhance the in vivo serum half-life of the administered compound, the compounds may be encapsulated, introduced into the lumen of liposomes, prepared as a colloid, or other conventional techniques may be employed which provide an extended serum half-life of the compounds.

The amount of compound administered to the patient will vary depending upon what is being administered, the purpose of the administration, such as prophylaxis or therapy, the state of the patient, the manner of administration, and the like. In therapeutic applications, compositions are administered to a patient already suffering from cancer in an amount sufficient to at least partially arrest further onset of the symptoms of the disease and its complications. An amount adequate to accomplish this is defined as “therapeutically effective dose.” Amounts effective for this use will depend on the judgment of the attending clinician depending upon factors such as the degree or severity of cancer in the patient, the age, weight and general condition of the patient, and the like. Preferably, for use as therapeutics, the compounds described herein are administered at dosages ranging from about 0.1 to about 500 mg/kg/day.

In prophylactic applications, compositions are administered to a patient at risk of developing cancer (determined for example by genetic screening or familial trait) in an amount sufficient to inhibit the onset of symptoms of the disease. An amount adequate to accomplish this is defined as “prophylactically effective dose.” Amounts effective for this use will depend on the judgment of the attending clinician depending upon factors such as the age, weight and general condition of the patient, and the like. Preferably, for use as prophylactics, the compounds described herein are administered at dosages ranging from about 0.1 to about 500 mg/kg/day.

The compounds of the invention may also be used in combination therapy with one or more additional biologically active agents. Virtually any suitable biologically active agent may be administered together with the ether lipids of the present invention. Such agents include but are not limited to antibacterial agents, antiviral agents, anti-fungal agents, anti-parasitic agents, tumoricidal agents, anti-metabolites, polypeptides, peptides, proteins, toxins, enzymes, hormones, neurotransmitters, glycoproteins, lipoproteins, immunoglobulins, immunomodulators, vasodilators, dyes, radiolabels, radio-opaque compounds, fluorescent compounds, receptor binding molecules, anti-inflammatories, antiglaucomic agents, mydriatic compounds, local anesthetics, narcotics, vitamins, nucleic acids, polynucleotides, etc. The entrapment of two or more compounds simultaneously may be especially desirable where such compounds produce complementary or synergistic effects. In particular, such biologically active agents include, but are not limited to, antineoplastic agents, antimicrobial agents, and hematopoietic cell growth stimulating agents.

For instance, in a recent study of ET-18-OCH₃ and a liposomal incorporated ET-18-OCH₃, it was found that apoptosis is triggered by this ether-lipid by induction of caspase activation through the release of cytochrome c in a Bcl-XL—sensitive manner but independently of the CD95 (APO-1/Fas) ligand/receptor system.²⁷⁻²⁸ CD95 is a surface membrane molecule involved in cell activation and apoptosis. It is expressed by a variety of hematopoietic cells, such as CD34+/CD38+ stem cells, myeloid cells and lymphocytes. Accordingly, the compounds according to the invention particularly when formulated in a liposome, may be used as an adjunct for the treatment of tumors in combination to myelosuppressive chemotherapeutic drugs and/or those that use the CD95-ligand/receptor system to trigger apoptosis.

As noted above, the compounds administered to a patient are in the form of pharmaceutical compositions described above. These compositions may be sterilized by conventional sterilization techniques, or may be sterile filtered. When aqueous solutions are employed, these may be packaged for use as is, or lyophilized, the lyophilized preparation being combined with a sterile aqueous carrier prior to administration. The pH of the compound preparations typically will be between 3 and 11, more preferably from 5-9 and most preferably from 7 and 8. It will be understood that use of certain of the foregoing excipients, carriers, or stabilizers will result in the formation of pharmaceutical salts.

As mentioned above, in a preferred embodiment, the compounds according to the invention will not aggregate platelets. With respect to avoiding platelet aggregation, various structural modifications to PAF have been studied, which provide guidelines for modifications that can be made to the antineoplastic ether lipids. For instance, PAF activity requires an ether linkage at the sn-1 position. Interestingly, unlike PAF, compounds having a sulfonate or sulfamoyl linkage at the sn-2 position, may not be susceptible to PLA₂ hydrolysis.³³ When the ether linkage is replaced with an ester linkage, the compound becomes susceptible to PLA inactivation, and no platelet aggregation is observed. It is thus likely that such compounds may survive enzymatic hydrolysis conditions in aiding prolong circulation and perhaps could yield potent and selective anti-neoplastic effects.

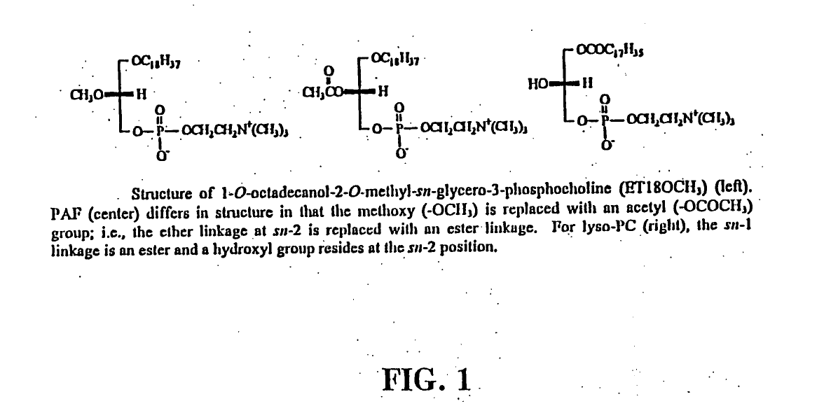

Further, the D isomer generally elicits no platelet aggregation. Additionally, when the acetyl group occupying the sn-2 position in PAF is replaced with another group, PAF activity may be decreased. In this regard, it was found that although replacement with propionyl doesn't decrease the activity, for every additional methylene unit added, the activity drops 10 fold compared to PAF. Additionally, when the acetyl group occupying the sn-2 position in PAF is replaced with a hydroxyl group (as in lyso PC shown in FIG. 1), there is no PAF activity. While not wishing to be bound by theory, the lack of PAF activity may be due to the susceptibility of the hydroxyl group to acylation. Finally, when the choline headgroup in PAF is replaced with another moiety, the platelet aggregation effect is diminished or non-existent.

The L isomer of ET-18-OCH₃ elicits a platelet aggregation response in dog whole blood, most likely due to its structural similarity to PAF. This response likely reflects an inherent promiscuity in the PAF receptor for dogs. This response can be blocked by PAF antagonists. Although no platelet aggregation has been observed using human blood from healthy volunteers, an aggregation event has been noted in platelet rich plasma (PRP) processed from the blood of healthy individuals, and in the whole blood of a few cancer patients. The physiochemical changes responsible for this have not yet been defined.

One way to avoid any potential hematological problems with ET-18-OCH₃ or other ether lipids has been to replace the L isomer with the D isomer, which doesn't elicit an aggregation response in dog whole blood. Particularly in cases where the D isomer exhibits roughly the same activity as the L isomer in terms of cytotoxicity and hemolytic activity, the D isomer is a likely candidate if a replacement is desired.

In a preferred embodiment of the invention, one or more of these factors are taken into account in order to produce a compound that does not exhibit a platelet aggregation effect.

In a further embodiment of the invention, the compounds will also not lyse red blood cells. If however, the compounds do lyse red blood cells, it is often possible to use a liposome carrier to minimize this effect. For instance, although ET-1S—OCH₃ has exhibited antitumor activity in several animal tumor models,⁸ its clinical use has been restricted by systemic toxic effects, e.g. hemolysis. In this regard, a stable liposomal system was developed that would incorporate ET-18-OCH₃ into the bilayer such that its release (exchange out) would be reduced. Using molecular monolayer studies of shape complementarity and formulation optimization, a lipid system which attained ideal packing between the host lipids and ET-18-OCH₃, resulting in a minimized hemolytic activity was determined. This a system, known as ELL-12, is now being evaluated in clinical trials,²³ Using such system, hemolysis has not presented a problem at doses exceeding those previously tried for the free compound.

In yet another embodiment of the invention, the antineoplastic ether lipids have desirable pharmacokinetic properties. For instance, it may be desirable to use a compound that is resistant to “rapid metabolism.” While not wishing to be limited by theory, lyso PC as shown in FIG. 1, is thought to be short lived because (1) the ester linkage is susceptible to phospholipase cleavage to produce GPC and (2) lyso PC's free hydroxyl is susceptible to acyltransferases. In contrast, ET-19-OCH₃ is thought to be resistant to the hydrolysis by membrane-associated phospholipases A1 and A2 (PLA1 and PLA2) due to its ether linkages with the sn-1 C18 chain and sn-2 methyl group.

Further, the choline and phosphocholine moieties are known targets for phospholipases C and D hydrolysis, which yields alkyl-glycerol and phosphocholine or phosphatidic acid and choline, respectively. One recent investigation, has shown that ET-18-OCH₃ at and above its cytotoxic concentrations did not inhibit phosphocholine-specific phospholipase C and phospholipase D, suggesting that ET-18-OCH₃ is not their primary target and could survive in biological membranes.²⁴ However, other studies revealed that ET-18-OCH₃ hexadecylphosphocholine (HPC) can be metabolized by PL-D, thus making an argument to replace the choline moiety to avoid PC specific PL-D hydrolysis.²⁵⁻²⁶

Likewise, phosphonocholine ET-18-OCH₃ analogs having a methylene residue instead of oxygen between the phosphorus and the glycerol moiety, could significantly help in providing less susceptibility to PL-C. Furthermore, modifying the headgroups with entities bulkier than choline may reduce susceptibility to choline-specific PL-D as well. This inaccessibility to phospholipases may allow these compounds to behave as long-acting anti-neoplastic agents.

In an embodiment of the invention, one or more of these factors are taken into account to produce novel ether lipid compounds that are stable to potential phospholipase degradation.

Specific embodiments of the invention will now be described through examples. The following synthetic and biological examples are offered to illustrate this invention and are not to be construed in any way as limiting the scope of this invention.

### EXAMPLES

In the examples below, the following abbreviations have the following meanings. If an abbreviation is not defined, it has its generally accepted meaning.

bd=broad doublet

bs=broad singlet

c=concentration

d=doublet

dd=doublet of doublets

ddd=doublet of doublets of doublets

DMF=dimethylformamide

DMSO=dimethyl sulfoxide

g=grams

hept.=heptuplet

J=coupling constant

m=multiplet

M=molar

max=maximum

mg=milligram

mM=minutes

mL=milliliter

mM=millimolar

mmol=millimole

N=normal

ng=nanogram

nm=nanometers

OD=optical density

q=quartet

s=singlet

sept=septuplet

t=triplet

THF=tetrahydrofuran

tlc=thin layer chromatography

μL=microliter

The antibodies were obtained from the following vendors: Transduction Laboratories, Lexington, Ky. (Raf-1, PKB/AKT); New England Biolabs Inc, Beverly, Mass. (phospho-MAP kinase and phospho-PKB/AKT); Santa Cruz Inc, Santa Cruz, CA (ERK-1, ERK-2); fetal bovine serum (FBS) from Hyaclone (Logan, Utah).

Additionally, the term “Aldrich” indicates that the compound or reagent used in the following procedures is commercially available from Aldrich Chemical Company, Inc., 1001 West Saint Paul Avenue, Milwaukee, Wis. 53233 USA; the term “Fluka” indicates the compound or reagent is commercially available from Fluka Chemical Corp., 980 South 2nd Street, Ronkonkoma, NY 11779 USA; the term “Lancaster” indicates the compound or reagent is commercially available from Lancaster Synthesis, Inc., P.O. Box 100, Windham, N.H. 03087 USA; and the term “Sigma” indicates the compound or reagent is commercially available from Sigma, P.O. Box 14508, St. Louis, Mo. 63178 USA.

Unless otherwise stated, all temperatures are in degrees Celsius.,

NMR spectra were recorded on an IBM-Bruker 200-MHz or a Bruker 400-MHz Spectrometer with Me₄Si as internal standard. Infrared spectra were recorded on a Perkin-Elmer 1600 FT spectrophotometer. Optical rotations were measured on a JASCO Model DIP-140 digital polarimeter using a 1-dm cell. Methylene chloride and pyridine were distilled from calcium hydride and barium oxide, respectively. Chloroform was distilled from P₂O₅. All other synthetic reagents were used as received unless otherwise stated.

In these synthetic methods, the starting materials can contain a chiral center and, when a racemic starting material is employed, the resulting product is a mixture of R,S enantiomers. Alternatively, a chiral isomer of the starting material can be employed and, if the reaction protocol employed does not racemize this starting material, a chiral product is obtained. Such reaction protocols can involve inversion of the chiral center during synthesis. Alternatively, chiral products can be obtained via purification techniques which separates enantiomers from a R,S mixture to provide for one or the other stereoisomer. Such techniques are well known in the art.

### Part I: Preparation of Compounds

I. Routes to the Synthesis of Phosphono-Ether Lipids

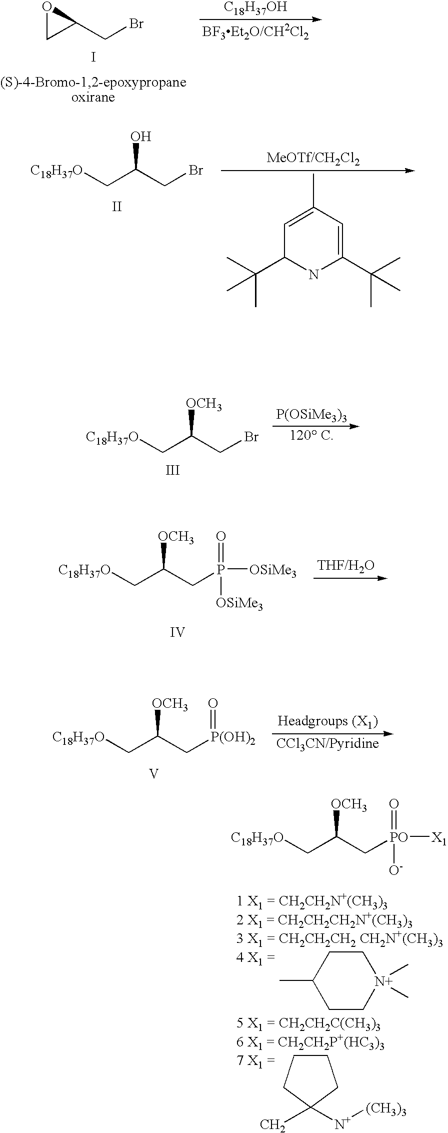

### 1-O-Octadecyl-2-hydroxy-4-bromobutane (II)

To a stirred solution of 1-octadecanol (36.0 g, 0.133 mol) and (s)-4-bromo-1,2-epoxybutane (I) (70.0 g, 0.133 mol) in 500 ml anhydrous CH₂Cl₂, was added BF₃ etherate (5 ml, 0.005 mol), and the reaction mixture was stirred for 18 hr under nitrogen atmosphere. After 18 hr, the solvent was removed under reduced pressure, the resulting white crude solid was purified via column chromatography by elution with 4% EtOAc in hexane to give white solid.

### 1-O-Octadecyl-O-methyl-4-bromobutane (III)

To 1-O-Octadecyl-2-hydroxy-4-bromobutane (II) (2.0 g, 4.75 mmol) in 40 ml of anhydrous CH₂Cl₂, was added 2,6-ditertbutyl-4-methyl-pyridine (4.9 g, 24 mmol). After 5 min of stirring, methyl triflate (3 ml, 26.6 mmol) was added and the reaction was continued at reflux at 40° C. for 16 hr. TLC was checked and solvent was removed under reduced pressure. The resulting residue was taken up in ethyl acetate and washed with 2M HCl (2×100 ml), 5% NaHCO₃, and dried over Na₂SO₄. Solvent filtration and removal under reduced pressure resulted in a white solid, which was purified on a silica gel column by elution with 2% EtOAc in hexane, yielding 1.4 g (68%) as flaky powder.

### 1-O-Octadecyl-2-O-methyl-butyl-4-phosphonic Acid (V)

A solution of compound (III) (1 g, 2.3 mmol) and excess of tris(trimethylsilyl) phosphite (5 ml) was heated at 125° C. for 24 hr to yield IV. The unreacted phosphite and bromotrimethylsilane were removed via vacuum distillation at ≦90° C. After the distillation, the crude bis-silylphosphite IV was cooled to room temperature and was subjected to hydrolysis in THF:H₂O (20 ml, 9:1) at room temperature for 12 hr to yield V. The crude waxy white product was dried and used in the next step without further purification.

### 2-Trimethylaminoethyl-1-O-octadecyl-2-O-methylbutane-4-phosphonate (1)

To a solution of 1-O-Octadecyl-2-O-methyl-butyl-4-phosphonic acid (V) (100 mg, 0.2 mmol) in anhydrous pyridine (15 ml), was added (2 ml, 20 mmol) of trichloroacetonitrile, and 2-trimethylaminoethanol tosylate (900 mg, 3 mmol). Solution was heated at 50° C. for 48 hrs under N₂ atmosphere. Solvent was removed under reduced pressure. Dark solid was dissolved in 9:1 THF:H₂O and passed through a TMD-8 ion-exchange resin column using the same eluents. Solvent removal under reduced pressure left a residue, which was purified via column chromatography by elution with gradients of CHCl₃, MeOH and H₂O. Compound 1 was lyophilized from cyclohexane to give 39 mg (38%), as white flaky solid.

Compounds 2-7 were prepared from the phosphonic acid (V) by the same procedure as described for 1.

### Part II: Methods for Evaluation of Compounds

The ether lipid compounds according to the invention may be screened by any acceptable method(s) used in the field. For example, the ether lipid compounds may be examined with respect to the ability of the new compounds 1) to aggregate platelets (i.e., mimic PAF), a specific toxicity to avoid or minimize, 2) to lyse red blood cells, a non-specific toxicity for which a liposome carrier may be needed, 3) to inhibit growth of tumor cells as a measure of activity, and 4) to inhibit growth of normal cell lines as compared to tumor cells, a measure of selectivity.

Candidates deemed suitable for in vivo testing were then synthesized in large scale quantities and some of this material was sent to the NCI's Developmental Therapeutics Program for a battery of growth inhibitory studies against various human tumor cell lines (60 cell lines, 9 different panels). The remainder of the material was used to assess in vivo efficacy using murine tumor models. Additionally, studies were conducted on the lead candidates to discern mechanism of action with particular emphasis on these agents apoptotic ability as measured by caspase 3 activity. The details (materials and methods) for these various as says are described further in the following discussion.

I. Platelet Aggregation Screening of New Ether Lipid (NEL) Derivatives

The following protocol measures platelet aggregation in whole blood utilizing a whole blood aggregometer from Chronolog Corp. The species most often used is dog since this species has consistently shown a strong platelet aggregation response in whole blood to L-ET-18-OCH₃, but any species, including human may be substituted. Briefly, whole citrated blood is diluted 1:2 with sterile saline and placed in a warm chamber with a mini stir bar. An electronic probe, measuring electrical resistance, is inserted in the sample. The aggregometer is calibrated and the baseline is observed to detect any spontaneous aggregation. The test sample is added and allowed to run for at least 6 minutes. If the sample is an agonist the platelets will start to aggregate and stick to the electronic probe causing resistance across the electrodes to increase. This resistance, in ohms, is measured 6 minutes post addition of sample. The test samples are run at 25, 100, 200, 400 and 800 uM and compared to 100 uM L-ET-18-OCH3.

Collection of Blood Sample for in vitro Platelet Aggregation Testing: Venous blood is collected in 4.5 mL. Vacutainer tubes containing 0.129 M sodium citrate using a 21G needle or larger. Blood is immediately mixed by gentle inversion 15-20 times and kept at room temperature. Dilution ratio is 1 to 9 (3.8% citrate solution:blood). One (2.0 mL) Vacutainer tube containing EDTA is also collected for platelet counts. Complete blood counts (CBC) are measured on the CDC Technologies Hemavet 1500 to ensure that the test subject's platelets are within normal range. Any vials of hemolytic blood or blood containing any clots should be discarded. Platelet aggregation testing must be completed within 3 hours of blood collection. After this time the ability of platelets to aggregate decreases.

Procedure for TLC ELL-12 dilutions: Test samples were provided from the Molecular Mechanisms Group either as powder or solutions. Cloudy solutions were warmed to ˜50° C. to dissolve and particles. Dilutions were prepared in PBS or saline at 40× concentration. (25 uL test sample are added to 1000 uL diluted blood (1:40)).

Procedure for in vitro Platelet Aggregation Test using CHRONO-LOG Aggregometer Model 560-CA: The following protocol was used:

1. Turn on aggregometer, aggrolink, monitor, computer and printer at least 15 minutes before testing to warm aggregometer to 37° C.

2. Double click on “AGGLINK” in Windows.

3. Set stir speed to 1000 for whole blood.

4. Set up small beaker with deionized water to clean impedance probes after each sample. Set up 2 plastic cuvettes with ˜1 mL of saline to store probes in warming chambers between tests. Set up 1 small plastic cuvette or test tube with ˜2 mL of saline to clean pipet after addition of test articles.

5. Click on “aggregometer”
Click on “test procedure” and set parameters
Procedure Name (Ether Lipid platelet aggregation test)
Channels=4
Duration=6:00 (min:sec)
Reagent (test article or L-EL control)
Concentration (25-800 uM)
Stirrer=1000
Gain=20/5 (20 ohms=5 blocks)
Enter OK to exit

6. Click on “aggregometer”
Click on “run test” and set parameters
Enter Subject Information
   Last Name=WB 1:2
   First Name: Subject
   ID#=time
   Hospital=N/A
Test Procedure (Edit if necessary)
Enter OK to exit

Make sure aggregometer temperature reads 37° C. before testing.

7. Place 1 mL plastic cuvettes into warming wells.

8. Add 1 disposable siliconized stir bar to each cuvette.

9. Add 500 uL saline to 2 cuvettes

10. Add 50.0 uL whole citrated blood to the two saline containing cuvettes. M-1000 positive displacement pipet is recommended to measure blood volume.

11. Warm diluted blood 5 min at 37° C. in warming wells.

12. Transfer diluted blood samples (in duplicate) to aggregation chambers and insert impedance probes. Close doors.

13. Calibrate each chamber (This must be done for each test run). Zero channels to baseline with zero knob. Hold in calibration button and adjust gain to 50%. Observe steady baseline for 1 minute. Recalibrate if necessary.

14. Open chamber door and add 25 uL test sample to each cuvette

15. Rinse capillary pipet piston with saline after each use.

16. Allow test to run at least 6 minutes.

17. Click “aggregometer” and then click on “stop test”

18. Click “Edit”

19. Click “set start & stop time.” Select channel 1 and hold down both mouse buttons while moving vertical start line to 3-5 seconds prior to sample injection (the stop time will automatically move to 6 min after start time).

20. Click done. Repeat step 15 and 16 to select channel 3.

21. Click “Edit”.

22. Click “compute slope & amplitude” and then check that both channels are set for 6 min run. Click OK. The aggregometer automatically calculates the ohms amplitude.

23. Click “file” and click “Save”

24. Click “File”

25. Click “close”

26. Remove impedance probe and place in beaker of deionized water

27. Gently remove any aggregated platelets from probe and place into warm saline prior to next test.

28. Discard test sample in biological waste.

29. Print out files Platelet Aggregation.

Platelet aggregation was assessed using dog whole blood, a system found to be highly sensitive as it has been demonstrated that the L-isomer of ET180CH3 invokes aggregation at relatively modest concentrations (but not the D-isomer): Results are shown in Table 2.

TABLE 2
Platelet aggregation of New Ether Lipid Analogs.

| | Relative Platelet Aggregation (Ohms) | | | | | Platelet |
|---|---|---|---|---|---|---|
| Compound | 25 μM | 100 μM | 200 μM | 400 μM | 800 μM | aggregation |
| L-ET18OCH₃ | 13 | 14 | 12 | nd | nd | +++ |
| D-ET18OCH₃ | 0 | 0 | 0 | nd | nd | − |
| 4(R) | 0 | 0 | 0 | 0 | 0 | − |

Platelet aggregation in dog whole blood was measured in Ohms. *All compounds were diluted from saline except 17-21 which, because of poor solubility, were given in DMSO (6S was given in 6% ethanol). Consequently, it is believed that much of the response noted for 17-21 was a DMSO response since DMSO alone evoked values similar to those recorded. For all experiments, 0.1 μM PAF and 100 μM ET-18-OCH₃ were included as positive controls.

When the number of methylenes in the headgroup was increased (i.e., compound 3R), no aggregation was noted indicating that those changes sufficiently altered binding to the PAF receptor.

II. In Vitro Hemolytic Activity Assessment

Before proceeding to in vivo testing, the hemolytic activity of the various compounds was examined to see how they compare to ET-18-OCH₃: a significantly more hemolytic agent might require a liposome for in vivo testing while one that is equal or less hemolytic would not (at least under the conditions established here for this screening study).

A. Hemolysis Assay with Washed Human Red Blood Cells

Venous blood was collected in 10 mL Vacutainer tubes containing EDTA using a 21G needle or larger. Blood was immediately mixed by gentle inversion 15-20 times and kept at room temperature. The blood was transferred from 1 EDTA tube to a 50 mL conical tissue culture tube and the volume was brought up to 50 mL with PBS.

The blood was centrifuged for 10 minutes at 1500 RPM. The supernatant was removed and the blood was resuspended up to 50 mL with PBS. Next the blood was centrifuged for 10 minutes at 1500 RPM.

The supernatant was removed, and 2.0 mL of packed red blood cells was carefully transferred, using positive displacement pipet, into a fresh conical tissue culture tube. Next, 48 mL PBS was added to achieve a 4% washed RBC solution.

Next, 25, 50, 100 and 200 uM stock solutions of test sample in phosphate buffered saline (PBS) as follows:

| Stock Solution | Test Sample | | PBS | |
|---|---|---|---|---|
| 200 uM | 200 uL of 20 mM | + | 19.8 | mL |
| 100 uM | 5 mL of 200 uM | + | 5 | mL |
| 50 uM | 1 mL of 100 uM | + | 1 | mL |
| 25 uM | 500 uL of 50 uM | + | 500 | uL |

Next, 0.5 mL of 4% washed RBC was added to 0.5 mL test sample dilutions (in duplicate). The final concentration of test sample was 50% of working stock solution.

The samples were capped or sealed with Parafilm and the samples were gently mixed. The blood was incubated at 37° C. in gentle rotating water bath for 30 minutes. The samples were centrifuged for 10 minutes at 1500 RPM. Next, 200 uL of supernatant was transferred to a cuvette and 1 mL deionized water was added. Absorbance was measured at 550 nm vs. a water blank. Next, H₁₀ and H₅₀ were determined by graphing Absorbance vs. Test Sample Concentration.

B. Hemolysis Assay with Whole Human Blood

Venous blood was collected in 10 mL Vacutainer tubes containing EDTA using a 21G needle or larger. The blood was immediately mixed by gentle inversion 15-20 times and kept at room temperature.

stock solutions of 20 mM test sample in phosphate buffered saline (PBS) were prepared as follows:

| Working Stock | | Test Sample | | PBS |
|---|---|---|---|---|
| 20,000 | uM | 100 uL of 20,000 uM | | |
| 10,000 | uM | 100 uL of 20,000 uM | + | 100 uL |
| 5,000 | uM | 100 uL of 10,000 uM | + | 100 uL |
| 2,500 | uM | 100 uL of 5,000 uM | + | 100 uL |
| 1,000 | uM | 100 uL of 2,500 uM | + | 100 uL |
| 500 | uM | 100 uL of 1,000 uM | + | 100 uL |

Then 270 uL of whole blood was aliquotted in duplicate mini test tubes using positive displacement pipet. Next, 30 uL of each working stock solution, in duplicate, was added to the whole blood. Next, 30 uL of PBS was added for background control. The samples were capped or sealed with Parafilm and gently mixed. The blood was incubated at 37° C. in gentle rotating water bath for 30 minutes. Final concentration of test sample was 10% of working stock solution.

Total hemolysis samples of 1% and 10% whole blood samples in deionized water were prepared as follows:

1:100=10 uL whole blood+990 uL deionized water

1:10=100 uL whole blood+900 uL deionized water

The samples were freeze thawed 3× in liquid nitrogen then water bath. The samples were then centrifuged 10 minutes at 1500 RPM. Next, 100 uL of supernatant was transferred to a cuvette and 1 mL deionized water was added.

The absorbance was read at 550 nm vs. water blank.

H10 and H50 were calculated by graphing Percent Total Hemolysis vs. Test Sample Concentration. The Percent Total Hemolysis=(average o.d of test sample)/(average o.d. of total hemolysis sample)×100

The test sample dilutions are shown below:

| Stock | | | Working | Final Conc |
|---|---|---|---|---|
| Conc. | uL Stock | uL PBS | Stock (uM) | (uM) |
| 200 | 500 | 0 | 200 | 100 |
| 200 | 450 | 50 | 180 | 90 |
| 200 | 400 | 100 | 160 | 80 |
| 200 | 350 | 150 | 140 | 70 |
| 200 | 300 | 200 | 120 | 60 |
| 200 | 250 | 250 | 100 | 50 |
| 200 | 225 | 275 | 90 | 45 |
| 200 | 200 | 300 | 80 | 40 |
| 200 | 190 | 310 | 76 | 38 |
| 200 | 180 | 320 | 72 | 36 |
| 200 | 170 | 330 | 68 | 34 |
| 200 | 160 | 340 | 64 | 32 |
| 200 | 150 | 350 | 60 | 30 |
| 200 | 140 | 360 | 56 | 28 |
| 200 | 130 | 370 | 52 | 26 |
| 200 | 120 | 380 | 48 | 24 |
| 200 | 110 | 390 | 44 | 22 |
| 200 | 100 | 400 | 40 | 20 |
| 100 | 180 | 320 | 36 | 18 |
| 100 | 160 | 340 | 32 | 16 |
| 100 | 140 | 360 | 28 | 14 |
| 100 | 120 | 380 | 24 | 12 |
| 100 | 100 | 400 | 20 | 10 |
| 50 | 100 | 400 | 10 | 5 |
| 50 | 50 | 450 | 5 | 2.5 |
| 25 | 40 | 460 | 2 | 1 |
| 25 | 0 | 500 | 0 | 0 |

Shown in Table 5 are the hemolytic activities for the ether lipids for which in vivo studies were planned.

TABLE 5
Hemolytic Activity of New Ether Lipids.

| | Washed Human RBCs | | Whole Human Blood | |
|---|---|---|---|---|
| Free | H₁₀ | H₅₀ | H₁₀ | H₅₀ |
| Compound | (μM) | (μM) | (μM) | (μM) |
| L-ET-18-OCH₃ | 11, 10.5 | 17, 15 | 600, 550, 700 | 2000, >2000 |
| 3R | 14, 12.5 | 18.5, 16.5 | 500 | >2000 |
| Liposome | | | | |
| Systems | | | | |
| L-ELL12 | 350 | >2000 | >2000 | >>2000 |
| H₁₀ and H₅₀ values are the concentrations at which the ether lipids produce 10% or 50% hemolysis, respectively, for assay details see Appendix II. D-ET18OCH3 historically produced the same values as the L isomer and is not shown here. Some experiments were repeated thus the additional entries. For the new liposome formulations, all liposomes were extruded to | | | | |
| # approximately 100 nm in mean diameter. Chol = cholesterol; DOPC = dioleoylphosphatidylcholine; DOPE-GA = dioleoylphosphatidylethanolamine-glutaric acid (glutaric acid is covalently attached via the headgroup nitrogen); CHS = cholesteryl-hemmisuccinate. | | | | |

III. In Vitro Growth Inhibition (GT₅₀)
A. Cell Line Maintenance

The following cell lines were selected from the cell bank for screening and GI₅₀ studies: MCF-7: human breast tumor, MCF-7/ADR: MCF-7 adriamycin resistant subline, HT-29: human colon carcinoma, A-549: human non-small cell lung cancer, NIH-3T3: mouse swiss embryo fibroblast and WI-38: human lung fibroblast, SKMEL-28: human melanoma, Lewis Lung: mouse lung carcinoma, DU-145: prostate carcinoma, B16F10: mouse melanoma, L1210: murine lymphocytic leukemia, P-88: murine leukemia, U-937: human histolytic lymphoma. Except HT-29, WI-38, NH-3T3 and Lewis Lung which were obtained from the American Type Culture Collection (Rockville, Md.) all the other cell lines were obtained from National Cancer Institute—Frederick Research Facility (Frederick; MD). All the cell lines were grown in RPMI-1640 medium containing 10% fetal bovine serum (FBS) except WI-38 and DU-145 which were grown in EMEM+10% FBS at 37° C., 5% CO₂ and 100% humidity and NIH-3T3, Lewis Lung, B16F10 and L1210 which were grown in DMEM containing 10% FBS (10% HS for L1210). All the adherent cell lines were detached: from the culture flasks by addition of 2-3 ml of 0.05% trypsin-EDTA. Thereafter, trypsin was inactivated by addition of 10 mL of 10% serum-containing RPMI-1640 medium. Cells were separated into a single-cell suspension by gentle pipetting action. Depending on the cell type, 3,000 to 10,000 cells were plated onto 96-well plates a day prior to the drug, treatment, in a volume of 100 μl per well.

B. Drug Treatment

The test compounds were made in-house and the compounds were dissolved PBS or saline at a stock concentration of approximately 20 mM, which is 400 times the desired final maximum test concentration. The stock solutions were then diluted with complete medium to twice the desired final concentration. 100 μl aliquots of each dilution was then added to the designated wells. After 3 days of incubation, cell growth was determined.

C. Sulforhodamine B (SRB) Assay

The SRB assay was performed as described by Monks, A., Scudiero, D., Skehan, P., Shoemaker, R., Paull. K., Vistica, D., Hose, C., Langley, J., Cronise, F.; and Vaigro-Wolff, A. Feasibility of a high-flux anticancer drug screen using a disperse panel of cultured human tumor cell lines. J Natl Cancer Inst, 83: 757-766, 1991 with minor modifications. Following drug treatment, cells were fixed with 50 μl of cold 50% (wt/vol) trichloroacetic acid (TCA) for 60 minutes at 4° C. The supernatant was discarded, and the plates were washed six times with deionized water and then air dried. The precipitate was stained with 100 μl SRB solution (0.4% wt/vol in 1% acetic acid) for 10 minutes at room temperature, and free SRB was removed by washing three times with 1% acetic acid, and the plates were then air dried. Bound SRB was solubilized with Tris buffer (10 mM), and the ODs were read using an automated plate reader (Bio-Rad, Model 3550-UV) at 490 nm. Background values were subtracted from the test data, and the data was calculated as a % of control. The GI₅₀ represents the concentration of test agent resulting in 50% of net growth compared to that of the untreated samples. In this assay, ODs were also taken at time 0 (the time the drugs were added) If the ODs of the tested samples were less than that of time 0, cell death had occurred. Percentage growth was calculated as described by Peters, A. C., Ahmad, I., Janoff, A. S., Plishkareva, M. Y., and Mayhew, E. Growth Inhibitory effects of liposome-associated 1-o-octadecyl-2-o-methyl-sn-glycero-3-phosphocholine. Lipids, 32:1045-1054, 1997. The raw optical density data was imported into an Excel spreadsheet to determine dose responses. Percentage growth was calculated as follows: (T−T₀)/(C−T₀)×100 where (T)=mean optical density of treated wells at a given drug concentration, (T₀)=mean optical density of time zero wells, and (C)=mean optical density of control wells, or if T<T₀ where cell killing has occurred, then percent death can be calculated as follows: (T−T₀/(T₀)×100. By varying drug concentration, dose response curves were generated and the GI-₅₀ values were calculated. The GI-₅₀ values for each experiment were calculated using data obtained from three duplicate wells on two separate plates. The mean GI-₅₀'s from each independent experiment.

D. Cell Growth Assay

To determine the Growth Inhibition in the suspension cell lines, cell numbers were directly counted instead of using the SRB assay which determines the total cell protein. One day prior to the drug treatment, 40,000 cells per well were seeded into 24-well plates in a volume of 0.5 mL. Stock solutions were diluted with complete medium to twice the desired final concentrations, and then 0.5 mL aliquots of each dilution were added to the designated wells. After 3 days incubation, cell growth was determined by counting cell number using a coulter counter (Z-M, coulter). Cell counts were also taken at time 0 and subtracted from the test results to give net growth. The GI-50 represents the concentration of test agent resulting in 50% of net growth compared to that of the untreated control samples.

F. Results

For growth inhibitory evaluation, five human tumor cell lines were used (U937; HT29; A549; MCF7; MCF7/ADR) and two normal fibroblast cell lines (NIH-3T3, murine; WI-38, human). For comparison, the activity of free L-ET-18-OCH₃ and D-ET-18-OCH₃ was examined.

TABLE 3
Growth Inhibition of Tumor/Normal Cells by New Ether Lipids.

| | GI50 (μM) | | | | | | |
|---|---|---|---|---|---|---|---|
| | | | | | | Normal Cell | |
| | Tumor Cell Lines | | | | | Lines* | |
| Compound | U937 | HT29 | A549 | MCF7 | MCF7/ADR | NIH-3T3 | WI38 |
| L-ET18OCH₃ | 1.0-1.5 | 5.5, 6.0 | 6.5-9.1 | 9.7-18.6 | 25.7->40 | 46.6 | 10-12.8 |
| D-ET18OCH₃ | 1.4 | 5.1 | 8.0 | 14.6 | 25.1 | 41.4 | 10-13.5 |
| 4(R) | nd | 2.6 | 7.2 | 28.5 | >40 | 58.5 | 9.8 |

*A human umbilical vein endothelial cell (HUVEC) line was also examined but it appeared to be very sensitive to ether lipids (for example, the GI₅₀ of L- and D-ET-18-OCH₃ were 1.2 and 1.4 μM) and was therefore not included for brevity.

As shown in Table 3, both L and D isomers of ET-18-OCH₃ gave essentially identical results with the order of sensitivity for the cells lines being U937>HT29>A549>MCF7>MCF7/ADR, NIH-3T3 (normal cell line). WI-38 cells were moderately sensitive to both ether lipids with GI50 values of 10-13 μM, which was 3-4 times lower than that for the NIH-3T3 cells at 41-47 μM.
IV. Measurement of DEVDase Activity

In a recent study, it was found that ELL12 triggers apoptosis by induction of caspase activation through the release of cytochrome c in a Bcl-xL—sensitive manner but independently of the CD95-(APO-1/Fas) ligand/receptor system [8]. To determine whether the new ether lipids produce their growth inhibitory effects via the same apoptotic mechanism as ELL12, and to correlate these results with activity, DEVDase activity, which is a specific correlate of Caspase 3 activity, was examined.

Suspension cells were seeded at density 3.2×10⁵ cells per mL in RPMI-1640 medium (Bio-Whittaker) supplemented with 10% heat inactivated FBS (Bio-Whittaker). Cells were pre-incubated overnight prior to treatment with the ether lipid compounds of the invention. At treatment time cell density was 5×10⁵ cells per mL. Cells were incubated with the ether lipid compounds of the invention for various periods of time, usually 6 hours. Cells were collected, washed with 1×DPBS and resuspended in 110 μl of Buffer A (10 nM HEPES-KOH, pH 7.4, 2 mM EDTA, 0.1% CHAPS, 5 mM DTT, 1:100 dilution of protease inhibitors cocktail (Sigma)—DTT and protease inhibitors should be added just before use). After 10 min incubation on ice in order to lyse cells, samples were frozen on dry ice/ethanol and kept at −20° C. until analysis.

At the time of analysis of DEVDase activity frozen pellets were kept on ice until thawed and after vigorous vortexing samples were centrifuged at 14,000 rpm for 6 min. Supernatant was transferred into another tube and 2 μl, in triplicates, were used for protein measurement using Bradford reagent (Bio-Rad).

Measurement of DEVDase activity was carried out in 100 μl volume, where 10 μg of protein was delivered in 50 μl of Buffer A and 40 μM of substrate Ac-DEVD-AMC was also delivered in 50 μl of Buffer A. All measurements were done in triplicates. Reaction was carried out for 1 hour and generation of fluorescent product of reaction (aminomethylcoumarin) was measured by reading fluorescence at 1ₑₘ 460 nm (1ₑₓ 360 nm). Changes in DEVDase activity were calculated after subtraction of background fluorescence of substrate incubated without proteins, and were expressed as percent of control (DEVDase activity in untreated cells). Average of DEVDase activity, calculated from few independent experiments, was used to calculate percent of L-ether lipid-induced DEVDase activity.

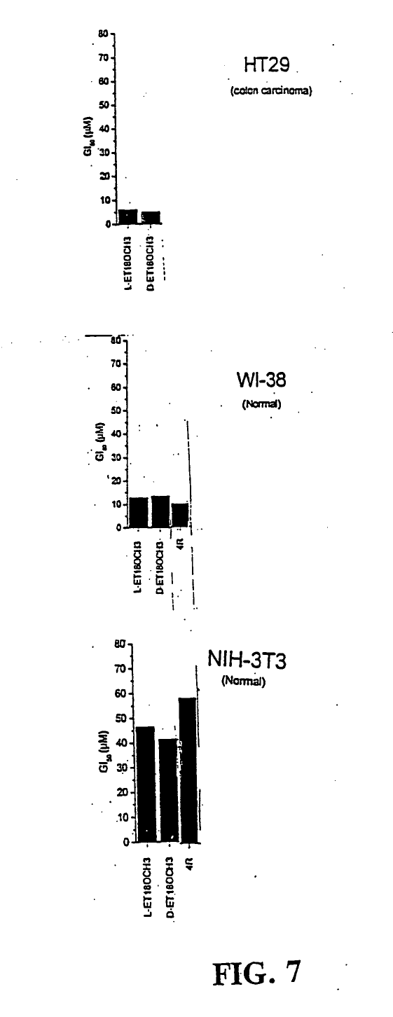

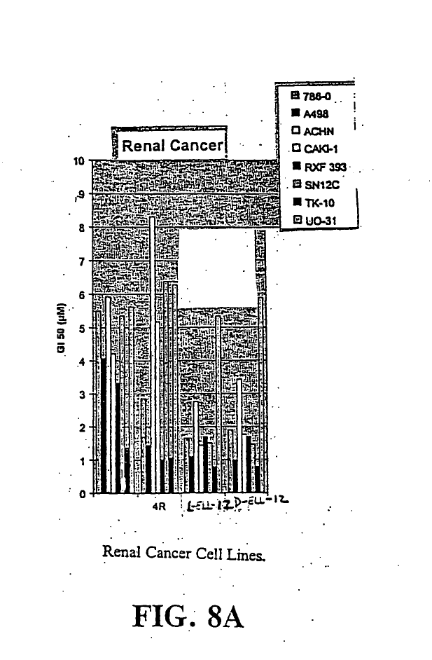

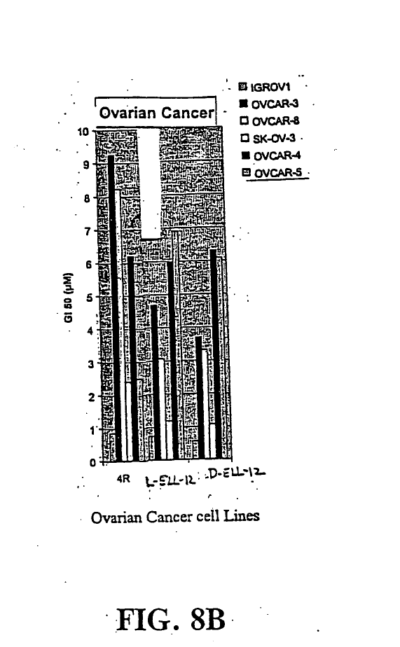

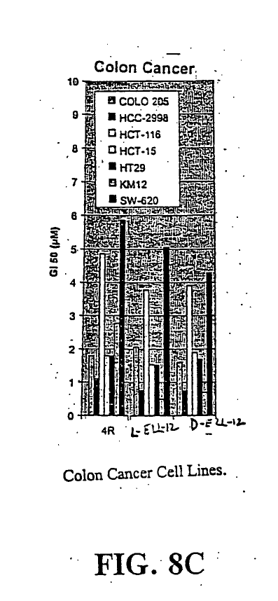

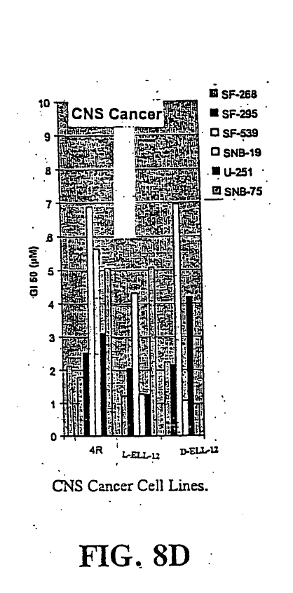

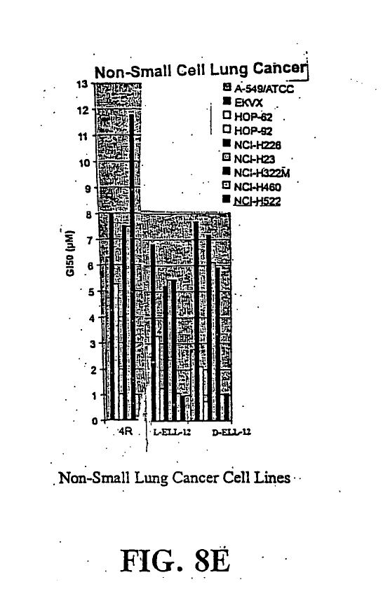

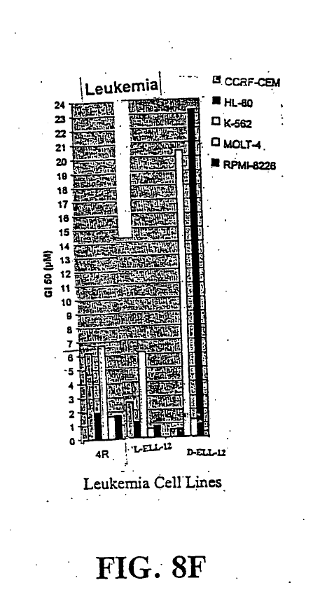

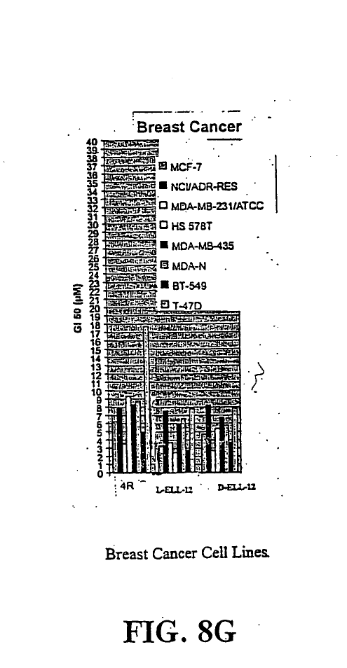

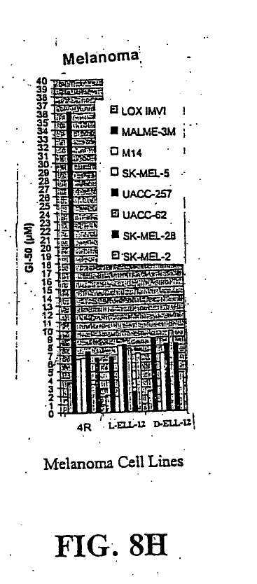

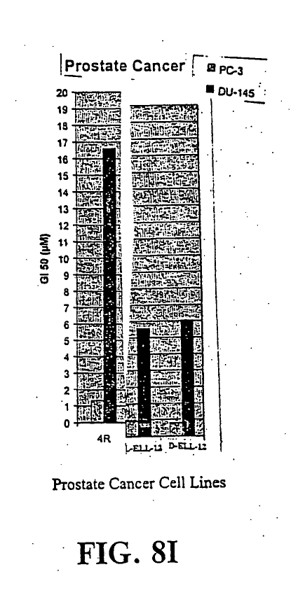

First, various conditions were examined to compare free ET18OCH3 and ELL12. As shown in FIG. 7, there is indeed a difference between liposomal compound and free drug. This difference is likely due to availability and, consistent with this notion, subsequent experiments showed that longer incubation times caused the differences to diminish (data not shown). However, the 6 hour time frame made for convenient testing and was used for comparative studies.

TABLE 7
DEVDase Activity of NELs in Jurkat T Cells at 6 Hours

| | % L-ET18OCH3 (DEVDase, 6 hrs) | | | |
|---|---|---|---|---|
| | [ether lipid], μM | | | |
| Lipid | 10 | 20 | 30 | N |
| L-ET18OCH₃ | 100 ± 8.8 | 100 ± 10.4 | 100 ± 9.3 | 7 |
| D-ET18OCH₃ | 79.3 ± 9.7 | 89.2 ± 12.2 | 92.1 ± 11.8 | 4 |
| L-ELL-12 | 66.1 ± 8.1 | 78.5 ± 10.8 | 71.3 ± 9.4 | 4 |
| D-ELL-12 | 56.5 ± 6.8 | 78.2 ± 10.5 | 85.5 ± 10.4 | 4 |
| 3R | 63.7 ± 7.7 | 73.9 ± 9.6 | 78.9 ± 9.4 | 3 |

N is the number replicate experiments.

As shown in Table 7, all of the new ether lipids exhibited DEVDase activity that was less than both isomers of ET-18-OCH₃.

V. In Vivo Toxicity and Therapeutic Methods

A. Toxicity

Intravenous (×1) or Oral (×1)

CDF1 mice (3/group) were administered a single intravenous or oral dose of the ether lipid to be tested. Mortality was recorded daily and body weights were recorded at least twice weekly for an observation period of 30 days.

B. Therapeutic

B16/F10 Murine Melanoma (iv./iv.)

Female C57/BL6 mice (5/group) were inoculated iv. with 5×10⁴ cells in 0.2 mL PBS (day 0). On days 10, 12, 14, 16, & 18 post-tumor inoculation, mice were treated iv. with the ether lipid to be tested; along with ELL-12 (L), D-EL, L-EL or Control (0.9% NaCl). Mice were sacrificed by carbon dioxide inhalation on day 22, lungs were excised, inflated and fixed with 10% Formalin. Lungs were counted “blind” for tumor nodules using a magnifier. The mean number of nodules per treatment group was determined.

P388 Murine Leukemia (ip./iv.)

Female CDF1 mice (7-8/group) were inoculated ip. with 1×10⁵ P388 cells in 0.5 mL PBS (day 0). Treatments were administered iv. on days 1, 3, 5, 7, & 9 post inoculation with the ether lipid to be tested, along with ELL-12 (L), L-EL, or Control. Mice were checked daily for mortality and the percent of survival was determined.

P388 Murine Leukemia (ip./ip.)

Female CDF1 mice (48/group) were inoculated ip. with 1×10⁵ P388 cells in 0.5 mL PBS (day 0). Treatments were administered ip. on days 1-10 post inoculation with ether lipid to be tested, along with ELL-12 (L), or Control or on days 1-8 post inoculation with ether lipid to be tested, along with D-EL, L-EL or Control (NaCl). Mice were checked daily for mortality and the percent survival was determined.

L1210 Murine Leukemia (ip./iv.)

Female DBA/2 mice (3-5/group) were inoculated ip. with 1×10⁵ cells in 0.5 mL PBS (day 0). Treatments were administered iv. on days 1, 3, 5, 7, & 9 post inoculation with ether lipid to be tested, along with D-EL, L-EL or Control (NaCl). Mice were checked daily for mortality and the percent survival was determined.

DU145 Human Prostate (sc./iv.)

Male SCID mice (5/group) were inoculated sc. with 2×10⁶ cells in 0.1 mL PBS (day 0) and the tumors were allowed to reach a volume of ˜250 mm³ at the start of treatment. Treatments were administered iv. on days 27, 28, 29, 30, & 31 with ether lipid to be tested, along with ELL-12 (D), ELL-12 (L), L-EL, or Control (NaCl). Tumors were measured with calipers and tumor volume (mm³) was calculated as (Length×(Width)²×p).

MX-1 Human Mammary (sc./iv.)

Female SCID mice (5/group) were inoculated sc. with 10 mg/0.1 mL tumor mince (day 0), and the tumors were allowed to reach a volume of ˜200 mm³ at the start of treatment. Treatments were administered iv. on days 13, 15, 17, 19, & 21 with ether lipid to be tested, along with ELL-12 (D). ELL-12 (L), L-EL, or Control (NaCl). Tumors were measured with calipers and tumor volume (mm³) was calculated as (Length×(Width)²×p).

Results

Ten compounds were also sent to NCI's Drug Discovery Program for screening against numerous human tumor cell lines (9 panels total: renal ovarian, colon, CNS, non-small cell lung, leukemia, breast, melanoma, and prostate). The data are shown in FIGS. 2a-i which also include data regarding D- and L-ET-18-OCH₃ (listed as L-EL and D-EL in the figures).

The invention has been described with reference to specific embodiments. Substitutions, omissions, additions and deletions may be made without departing from the spirit and scope of the invention defined in the appended claims. From the foregoing description, various modifications and changes in the composition and method will occur to those skilled in the art. All such modifications coming within the scope of the appended claims are intended to be included therein.

All of the above publications, patents and patent applications are herein incorporated by reference in their entirety to the same extent as if each individual publication, patent or patent application was specifically and individually indicated to be incorporated by reference in its entirety.

The claimed invention is:

1. An ether lipid having formula (I), or a pharmaceutically acceptable salt, isomer or prodrug thereof: 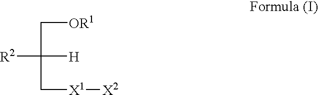
wherein:
R¹ is selected from the group consisting of alkyl, alkenyl and alkynyl;
R² is selected from the group consisting of —OR³;
R³ is selected from the group consisting of C₁₋₄ alkyl and H;
X¹ is selected from the group consisting of —OSO₂(CH₂)ₘ—, —OSO₂NR⁴(CH₂)ₘ, 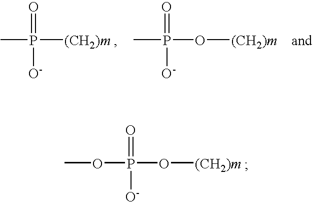
X² is selected from the group consisting of: 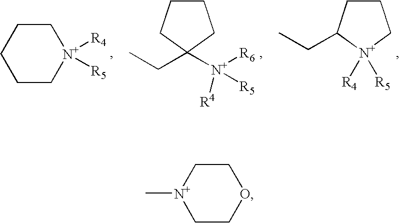
—CH₂(CH₂)ₚN⁺R⁴, R⁵, R⁶ and —CH₂(CH₂)ₚP⁺R⁷, R⁸, R⁹,
R⁴, R⁵ and R⁶ are each independently selected from the group consisting of H and C₁₋₁₀alkyl.
R⁷, R⁸ and R⁹ are each independently a C₁₋₃alkyl group; and
and m and p are each independently 0 or an integer from 1-10.

2. An ether lipid of claim 1, wherein R¹ is represented by Y¹Y², wherein:
Y¹ is (CH₂)n₁(CH═CH)n₂(CH₂)n₃(CH═(CH)n₄(CH₂)n₅(CH═CH)n₆(CH₂)n₇ (CH═CH)n₈(CH₂)n₉ the sum of n₁+2n₂n₃+2n₄ is equal to n₅+2n₆+n₇+n₈+n₉ is an integer of from 3 to 23, n₁ is zero or an interger of from 1 to 22, n₃ is zero or an integer of from 1 to 19, n₅ is zero or an integer of from 1 to 16, n₇ is zero or an integer of from zero to 16, n₉ is zero or an integer of from 1 to 10, and each of n₂, n₄, n₆ and n₈ is independently zero or 1; and
Y₂ is —CH₃ or —CO₂H.

3. An ether lipid of claim 2, wherein R¹ is selected from the group consisting of —C₁₈H₃₇ and —C₁₆H₃₃.

4. An ether lipid of claim 3, wherein R¹ is —C₁₁H₃₇.

5. An ether lipid of claim 4, wherein:
R³ is Me.

6. An ether lipid of claim 1, wherein X¹ is SO₂(CH₂)ₘ—and

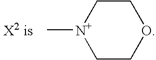

7. An ether lipid of claim 6, wherein m is an integer from 1-5.

8. An ether lipid of claim 1, wherein 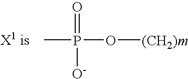
and X² is
—CH₂(CH₂)ₚN⁺R⁴R⁵R⁶.

9. An ether lipid of claim 8, wherein m is 0 and p is an integer from 1-8.

10. An ether lipid of claim 8, wherein R⁴, R⁵ and R⁶ are each a methyl group.

11. An ether lipid of claim 1, wherein 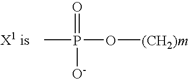
and X² is —CH₂(CH₂)ₚP⁺R⁴R⁵R⁶.

12. An ether lipid of claim 11, wherein m is 0 and p is an integer from 1-8.

13. An ether lipid of claim 11, wherein R⁴, R⁵ and R⁶ are each a methyl group.

14. An ether lipid of claim 1, having the following chemical formula: 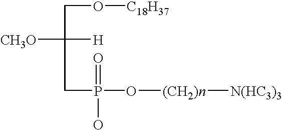
wherein n is 2, 3, 4, 5, or 6.

15. An ether lipid of claim 1, having the following chemical formula:

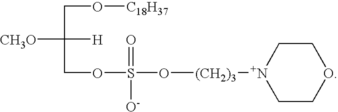

16. An ether lipid of claim 1, having the following chemical formula:

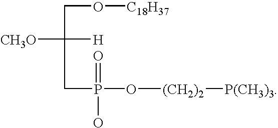

17. An ether lipid of claim 1, wherein the ether lipid is optically active.

18. An ether lipid of claim 1, wherein the ether lipid is the D enantiomer.

19. An ether lipid of claim 14, wherein the ether lipid is the D enantiomer.

20. An ether lipid of claim 15, wherein the ether lipid is the D enantiomer.

21. An ether lipid of claim 16, wherein the ether lipid is the D enantiomer.

22. A pharmaceutical composition comprising a pharmaceutically effective amount of an ether lipid of claim 1 or a pharmaceutically acceptable salt, isomer or prodrug thereof, and a pharmaceutically acceptable carrier.

23. A pharmaceutical composition comprising:
(a) a liposome, emulsion or mixed miscelle carrier and
(b) a pharmaceutically effective amount of an ether lipid of claim 1 or a pharmaceutically acceptable salt, isomer or prodrug thereof.

24. A liposome comprising an ether lipid of claim 1 or a pharmaceutically acceptable salt, isomer or prodrug thereof.

25. A method of treating a mammal afflicted with a cancer which comprises administering to the mammal a therapeutically effective amount of the pharmaceutical composition of claim 22 comprising from about 0.1 mg of the ether lipid per kg of the body weight of the mammal to about 1000 mg per kg.

26. A method of claim 25, wherein the cancer is selected from the group consisting of lung cancers, brain cancers, colon cancers, ovarian cancers, breast cancers, leukemias, lymphomas, sarcomas and carcinomas.

27. The method of claim 25, comprising administering to the mammal an additional biologically active agent.

28. The method of claim 27, wherein the additional biologically active agent is selected from the group consisting of antineoplastic agents, antimicrobial agents, and hematopoietic cell growth stimulating agents.
